(19) United States
(12) Patent Application Publication
(10) Pub. No.: US 2026/0069422 A1
(43) Pub. Date: Mar. 12, 2026

(54) TIBIA IMPLANT

(71) Applicant: Aesculap AG, Tuttlingen (DE)

(72) Inventors: Berna Richter, Muehlheim (DE); Julius Dohm, Kronberg (DE); Thomas Grupp, Denkingen (DE)

(21) Appl. No.: 19/318,547

(22) Filed: Sep. 4, 2025

(30) Foreign Application Priority Data
Sep. 11, 2024 (DE) 10 2024 126 035.1

(51) Int. Cl. A61F 2/38; A61F 2/30; A61L 27/06; A61L 27/30

(52) U.S. Cl. CPC A61F 2/389; A61F 2/30771; A61L 27/06; A61L 27/306; A61F 2002/30176; A61F 2002/30321; A61F 2002/30324; A61F 2002/30838; A61F 2002/30891; A61L 2420/06; A61L 2420/08

(57) ABSTRACT

A tibia implant for joint replacement has a plateau section and an anchor section projecting from a tibia-facing side of the plateau section. The anchor section is insertable into a channel in a tibia bone. The plateau section has a first surface structure that is a porous open-pore surface structure with bridges, webs or wall regions that can be gripped from behind in the axial direction on the tibia-facing side that comes into contact with tibia bone tissue. The open-pore surface structure has a first roughness. The anchor section is connected to the plateau section in a first axial anchor region on the circumferential side has a surface structure with a second roughness that is lower than the first roughness. The anchor section has a second axially free-ending axial anchor region with a smooth surface in the axial direction adjacent to the first axial anchor region.

### CROSS-REFERENCE TO RELATED APPLICATIONS

This application claims priority under 35 U.S. C. § 119 to German Application No. 10 2024 126 035.1, filed on Sep. 11, 2024, the content of which is incorporated by reference herein in its entirety.

### FIELD

The present disclosure relates to a tibia implant for joint replacement, which is made of metal alloy in an additive manufacturing method, in particular according to standard ASTM 52900:2022-03, in particular by metallic 3D printing, with a plateau section on which a bearing region for articulating condylar joint surfaces is provided on the side facing away from the tibia and, in particular, a meniscus replacement part can be arranged, and with a pin-shaped or keel-shaped anchor section projecting on the tibia-facing side of the plateau section and extending away from it in an axial direction, which can be inserted in an axial direction into a channel prepared for this purpose in a tibia bone.

### BACKGROUND

In the field in question, a distinction is made between implants that are typically cemented into a channel prepared for this purpose in the tibia and those that are inserted without cement and ultimately anchored in the tibia by the growth of tibial bone tissue. Naturally, both methods aim to achieve a permanent secure fit of the tibial implant in the tibia. However, in individual cases, it may be necessary to remove the implant from the tibia and replace it with a new one.

Numerous suggestions have been made regarding the design of the various surface regions of an implant in order to achieve a secure fit for both cemented and cementless implants. For example, US 2024/0041605 A1 suggests providing the shaft of an arthroscopic implant with circumferential regions of varying porosity and roughness with lattice structures that can be gripped from behind.

### SUMMARY

The present disclosure is based on the task of further developing a tibia implant of the type mentioned above in such a way that a permanent secure fit in the tibia can also be achieved by means of cementless implantation, wherein, if desired, it should also be possible to remove the implant without damaging the tibia.

To solve this problem, based on a tibia implant of the type mentioned above, the present disclosure proposes that the plateau section on the tibia-facing side which comes into contact with tibia bone tissue has a three-dimensionally porous, open-pore surface structure with bridges, webs, or wall regions that can be gripped from behind in the axial direction, wherein the three-dimensionally porous, open-pore surface structure has a first roughness
that the pin-shaped or keel-shaped anchor section adjacent to the plateau section in a first axial anchor region has, on the circumferential side, a surface structure free of undercuts in a radial direction, i.e., free of bridges and bridge-forming webs that can be gripped from behind, with a second roughness that is less than the first roughness, and
that the pin-shaped or keel-shaped anchor section has, in the axial direction adjacent to the first axial anchor region, a second axially free-ending axial anchor region with a smooth surface.

The design of the tibia-facing side of the plateau section, with a surface structure featuring bridges, webs, or wall regions that can be gripped from behind in the axial direction, tibial bone tissue can not only grow onto the metal surface, but also into the three-dimensional porous surface structure, thus holding the implant body firmly to the tibia in the implantation direction, i.e., in the axial direction. In the event of extraction, a resection cut can essentially be made in a radial direction, i.e., perpendicular to the axial or transplantation direction, using a saw blade in order to sever this stable connection for the purpose of extracting the implant, which is easily possible in a surgical situation.

Due to the fact that, axially adjacent to the plateau section in the first axial anchor region, a surface structure free of undercuts in the radial direction is provided on the circumferential side, i.e., a surface structure free of bridges or bridge-forming webs that can be gripped from behind, whose second roughness is lower than the first roughness of the aforementioned three-dimensionally porous surface structure, the tibia bone tissue that then forms can only grow onto this circumferential surface structure; in the absence of three-dimensional porosity and bridges and webs that can be gripped from behind, however, ingrowth into regions, tunnels, or pathways that can be gripped from behind is not possible. It has been established that growing bone tissue differs in its biological structure from bone tissue growing into porous structures and that the former is easier to remove under shear stress than the latter. Nevertheless, even on surface structures that are free of undercuts, only growing bone tissue can form a considerable adhesive force and a permanent connection with the structure, so that a permanent secure fit can also be achieved in this way, which is able to withstand the stresses occurring during operation of the implant without the joint replacement implant becoming loose.

It was further found that it is advantageous if a second distal anchor region, which is connected to the first axial anchor region in the axial direction, is smooth, because this facilitates the insertion of the anchor section into the channel prepared for this purpose in the tibia; in addition, this smooth surface design prevents tibial bone tissue from being scraped off when the anchor section is inserted into the prepared channel, thus preventing the dimensional accuracy of the channel from being altered in an unpredictable manner. Furthermore, it was shown that, with this smooth design, the implant can also be removed more atraumatically in the second axial anchor region if desired.

When the present application refers to a smooth surface formation on the implant or an implant region, this means that there is no three-dimensionally varying surface structure with elevations, recesses, or webs resulting from the additive manufacturing method, in particular from 3D printing, but rather that the additive manufacturing method was carried out in a volume-filling manner so that a closed surface is created which only exhibits a low surface roughness inherent to additive manufacturing, which is accordingly also significantly lower than the second roughness of the first axial anchor region. Following additive manufacturing, the smooth, uniform surface can also be polished if desired.

Furthermore, it should be explained that a surface is less rough within the meaning of the present application than another surface if it is less rough than another surface in terms of the roughness parameter Ra determined in accordance with DIN/ISO 21920-3.

It further proves advantageous if the three-dimensional porous surface structure has a depth extension extending from a surface or envelope area enclosing the surface structure of at least 1.5 mm, in particular at least 1.8 mm, in particular at least 2.0 mm, in particular at least 2.5 mm, in particular at most 4.0 mm, in particular at most 3.8 mm, in particular of at most 3.5 mm, in particular of at most 3.0 mm. A depth extension of 2.0 to 3.5 mm is preferred. Within the regions mentioned, satisfactory cement-free anchoring of the plateau section of the tibia implant can be achieved through the ingrowth of bone tissue into the three-dimensional porous open-pore surface structure.

It also proves advantageous and readily achievable by additive manufacturing technology that the three-dimensional porous open-pore surface structure is formed by a continuous web structure forming a three-dimensional lattice and that a web diameter is at least 0.5 mm, in particular at least 0.6 mm, in particular at least 0.7 mm, in particular at least 0.9 mm, in particular at most 1.1 mm, in particular at most 1.0 mm, in particular at most 0.9 mm.

It is also advantageous if the three-dimensional porous open-pore surface structure has a pore size of at least 0.8 mm, in particular at least 0.9 mm, in particular at least 1.0 mm, in particular at most 1.4 mm, in particular at most 1.3 mm, in particular at most 1.2 mm, which is the diameter of a sphere that can be accommodated in the pores.

In further development of the present disclosure, it proves advantageous if the circumferential surface structure of the first axial anchor region, which is free of undercuts in the radial direction, has a significantly smaller depth extension starting from a surface or envelope area enclosing the surface structure than the three-dimensional porous open-pore surface structure in the plateau section. Based on a surface structure enclosing the surface, it is at least 0.2 mm, in particular at least 0.3 mm and at most 0.9 mm, in particular at most 0.8 mm, in particular at most 0.7 mm.

It further proves advantageous if the circumferential surface structure of the first axial anchor region, which is free of undercuts in the radial direction, has structures rising from a base area of the anchor region. Here, the base area can form a smooth circumferential area of the first axial anchor region relative to the structures rising from it, from which these structures then rise like mountain structures.

Furthermore, it may be advantageous if the surface structure, which is free of undercuts in the radial direction, of the first axial anchor region, when viewed in the radial direction, has meandering, rising structures.

It may also prove advantageous if the rising structures comprise a plurality of flat faceted areas that are adjacent to one another via edges. In particular, the rising structures can be completely formed or delimited by such flat faceted areas. The growth of bone tissue on flat faceted areas has proven effective in the application in question here.

It also works well if the meandering, rising structures essentially delimit flat or smooth regions of a base area between them.

Furthermore, it proves advantageous if neighboring meandering, rising structures touch each other and delimit areas between them that are free of elevation or less elevated. When viewed in a radial direction, the rising structures form a regular, periodic pattern and a flat, net-like structure, with openings formed by the areas that are free of elevation or less elevated.

However, it can also be advantageous if neighboring meandering, rising structures are spaced apart from each other so that strip-shaped, contiguous areas that are free of elevation or less elevated are delimited between them.

Furthermore, it may also prove advantageous if the radially undercut-free surface structure of the first axial anchor region has a plurality of individual island-like rising structures spaced apart from one another. In such cases, contiguous areas that are free of elevation or less elevated are formed or delimited between these structures.

It is also suggested that areas that are free of elevation or less elevated between rising structures should be smooth. In this case, the structures that rise up are like mountain structures, rising from the flat valleys between them.

Since then, cobalt-chromium alloy has typically been used as the material for cement-free implants, but this has proven problematic in terms of compatibility. In this respect, it is advantageous that it has been established that an implant of the type described here can be manufactured from titanium or titanium alloy and that, nevertheless, both the ingrowth of bone tissue into structures that can be gripped from behind, pores, tunnels, and pathways on the one hand, and the growth of bone tissue onto less complex surface structures without bridges and tunnels on the other hand, lead to a sufficient secure fit.

In a further development of the present disclosure, it is also possible for surface regions of the plateau section that are not in contact with tibial bone tissue, i.e., circumferential regions in contact with soft tissue or surface regions in contact with a meniscus replacement part, to be smooth. It has been found that implant surfaces in contact with soft tissue cause problematic tissue irritation and can therefore have a very negative effect. In this respect, it is also proposed that smooth surface regions of the plateau section that are not in contact with tibial bone tissue should, at least in some regions, have a coating that further reduces the surface roughness of these smooth surface regions. Such a coating, in particular for surface regions of knee joint implants that come into contact with soft tissue, was described in patent application DE 2023 114 759 by the applicant. The formation, in particular the multi-layered formation, of this coating is explained in this unpublished patent application DE 2023 114 759, so that for disclosure purposes the relevant content is incorporated by reference into the present application:

Accordingly, it proves particularly advantageous if the coating comprises a ceramic surface, especially made of or based on zirconium nitride.

It is also advantageous if the coating is multi-layered and bonded to the implant component via an adhesion-promoting layer, in particular one based on cobalt-chromium or titanium.

It is also a plus if the coating has layers based on cobalt-chromium and/or chromium nitride (CrN) and/or chromium carbonitride (CrCN) and/or zirconium nitride (ZrN).

It is also advantageous if the coating has a top layer based on zirconium nitride (ZrN) and inner layers based on chromium nitride (CrN) or chromium carbonitride (CrCN), wherein inner layers can alternate based on chromium nitride (CrN) and chromium carbonitride (CrCN).

### BRIED DESCRIPTION OF THE DRAWINGS

Further features, details, and advantages of the present disclosure are apparent from the drawings and the following description of a preferred embodiment of the tibia implant according to the present disclosure.

FIG. 1 shows a perspective view of a tibia implant according to the present disclosure;

FIG. 2 shows a three-dimensional porous surface structure on the tibia-facing side of a plateau section of the tibia implant according to FIG. 1;

FIG. 3 shows a schematic view of a surface structure free of undercuts in the radial direction in a first axial anchor region of a pin-shaped or keel-shaped anchor section of the tibia implant according to FIG. 1; and

FIGS. 4a-4d show schematic views of the extension of rising structures in the surface structure that is free of undercuts in the radial direction in the first axial anchor region.

### DETAILED DESCRIPTION

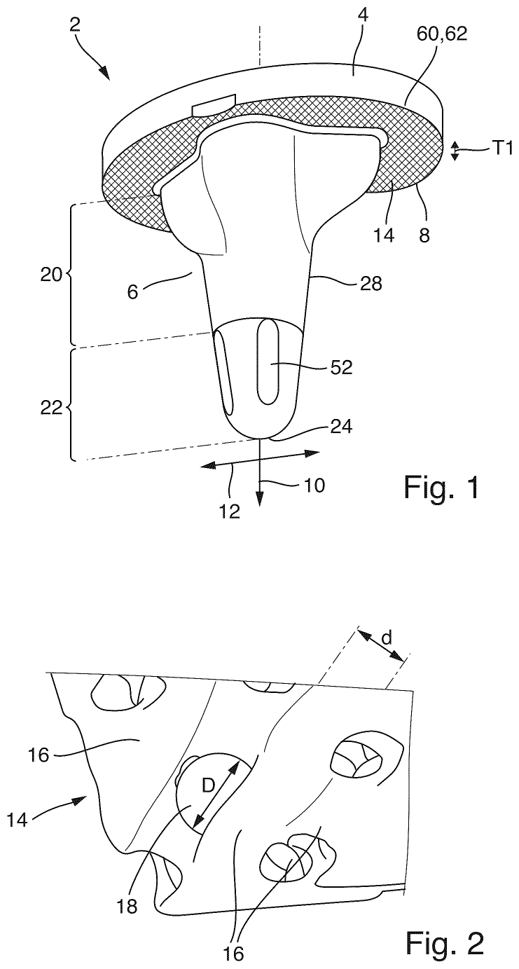

FIG. 1 shows a tibia implant 2 designed according to the present disclosure for a knee joint replacement prosthesis. It is manufactured using an additive manufacturing method, in particular by metallic 3D printing, and comprises a plateau section 4 and a pin-shaped or keel-shaped anchor section 6 projecting from the tibia-facing side of the plateau section 4. The pin-shaped or keel-shaped anchor section 6 extends from a tibia-facing side 8 of the plateau section 4 in an axial direction 10, which also forms an implantation direction of the tibia implant 2. A radial direction for this is indicated with reference symbol 12. In a manner not shown, the plateau section 4 facing away from the tibia can form a bearing region for articulating condylar joint surfaces; in particular, a meniscus replacement part can be arranged there in a known and therefore not shown manner. FIG. 1 shows the tibia implant 2 at an angle to the axial direction 10 from below, i.e., with a view of the tibia-facing side 8 of the plateau section 4.

This tibia-facing side 8 of the plateau section 4, which comes into contact with tibia bone tissue, is formed with a three-dimensional porous open-pore surface structure 14. This surface structure 14 has bridges, webs, or wall regions that can be gripped from behind in the axial direction 10 and in other directions. This three-dimensional porous open-pore surface structure 14 is shown greatly enlarged in FIG. 2, where a web structure forming a three-dimensional lattice can be seen, which is made up of lattice-like connected webs 16, wherein these webs 16 form bridges, webs, or wall regions that can be gripped from behind in several directions. This surface structure 14 and its webs 16 can thus, in a sense, be gripped from behind by ingrowing tibial bone tissue in every direction, i.e., in the axial direction 10 and in the radial direction 12, whereby an intimate connection of tibial bone tissue to the tibia-facing side 8 of the plateau section 4 is formed and a secure fit of the tibia implant 2 in the tibia can be achieved. The sphere 18 shown in FIG. 2 serves to indicate and dimension pore sizes within the three-dimensional porous surface structure 14. It is not part of the structure, but is only used for illustrative purposes. The webs 16 of the web or lattice structure result in an initial roughness, expressed in Ra, of the surface structure 14. It has a depth extension T1 extending from an envelope area applied from the outside and touching the surface structure to the three-dimensionally dense metallic base of the plateau section 4, as indicated in the introductory description. The same applies to the web diameter d and a pore size D (both indicated in FIG. 2).

Starting from the tibia-facing side 8 of the plateau section, the pin-shaped or keel-shaped anchor section extends in the axial direction 10. It comprises a first axial anchor region 20 and, adjoining this, a second axial anchor region 22, which also forms a distal end 24 of the entire pin-shaped or keel-shaped anchor section 4.

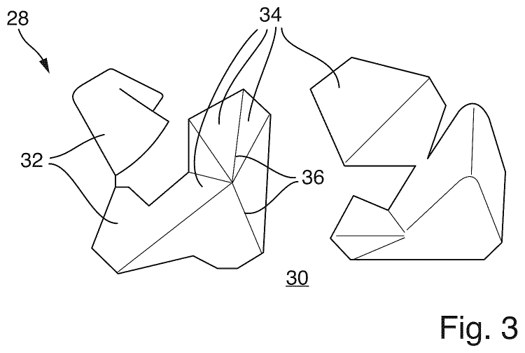

As indicated in FIG. 1, the first axial anchor region 20 comprises, on the circumferential side, a surface structure 28 that is free of undercuts in the radial direction 12 and free of bridges and bridge-forming webs. The structure of this surface structure 28, which is free of undercuts in the radial direction 12, is shown in a highly schematic form in FIG. 3, viewed in the radial direction 12, i.e., looking toward an outer circumference of the first axial anchor region 20. In this example shown in FIG. 3, the surface structure 28 is formed by structures 32 extending or rising from a base area 30 of the first axial anchor region 20. In the exemplary and preferred embodiment, the base area 30 is smooth and flat relative to the structures 32. In the example shown, the rising structures 32 are formed and delimited by a plurality of flat faceted areas 34, which are connected to one another by essentially straight edges 36. The surface structure 28 has a depth extension T2 (not shown in the figures) extending from an envelope area applied from the outside and touching the elevations 32 to the three-dimensionally dense metallic base or the base area 30 of the first axial anchor region 20, as indicated in the introductory description.

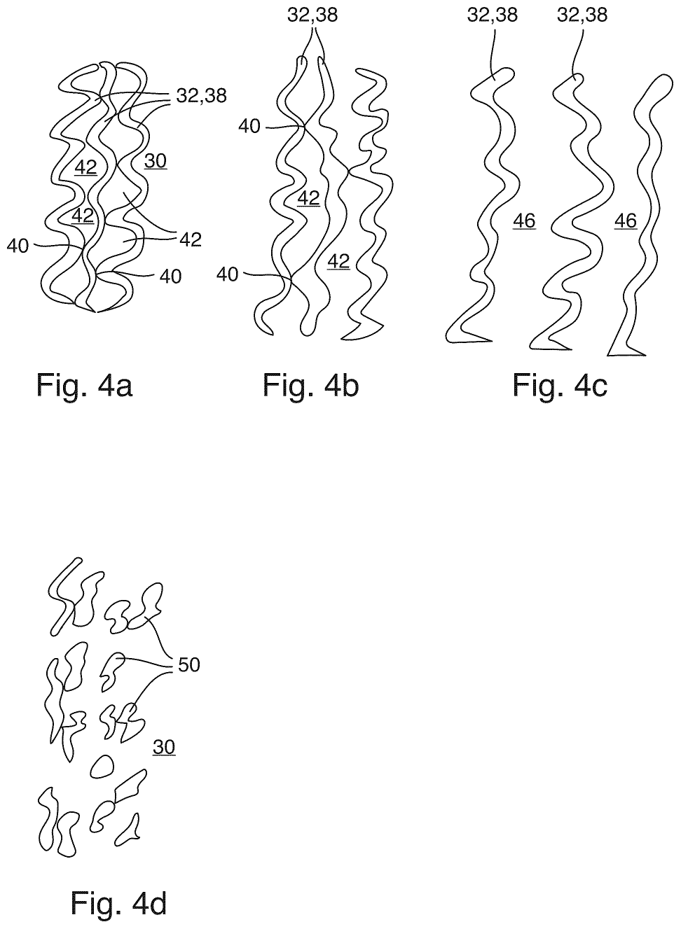

FIGS. 4a through 4d show examples in a greatly simplified form, i.e., without any indication of a three-dimensional structure, merely in a two-dimensional top view, of the arrangement of rising structures 32 of the surface structure 28 that is free of undercuts in the radial direction 12. FIGS. 4a through 4d show, in plane view of the first axial anchor region 20 in the radial direction 12, various embodiments of the course and extension of rising structures 32, wherein the three-dimensional design, such as delimiting formed by faceted areas 34, as shown in FIG. 3, is neither necessarily provided nor shown in FIGS. 4a through 4d. It is more a question of the arrangement and extension of the structures that rise up in the first axial anchor region 20. FIG. 4a shows rising structures 32 that meander back and forth and are therefore fan-shaped or zigzag-shaped, and are designated by reference symbols 38. In FIG. 4a, meandering structures arranged next to each other form 32 contact points 40, so that mesh-like areas 42 that are free of elevation or less elevated are enclosed or delimited between adjacent structures 32. As already mentioned above, these can be formed from smooth regions of a base area 30 of the first axial anchor region 20.

In the illustration shown in FIG. 4b, adjacent meandering structures 32, 38 have only a small number of contact points 40, so that elongated areas 42 that are free of elevation or less elevated are delimited between structures 32, 38. This could give the impression of a flat, torn net.

In FIG. 4c, adjacent meandering rising structures 32, 38 are spaced apart without touching each other in such a way that strip-shaped, contiguous areas 46 that are free of elevation or less elevated are formed between them.

Finally, FIG. 4d illustrates a surface structure 28 in which a plurality of island-like, spaced-apart, rising structures 50 are formed, which in turn rise up in particular and preferably from a smooth base area 30 of the first axial anchor region 20.

The second axial anchor region 22 adjoining the first axial anchor region 20 does not have a three-dimensionally porous surface structure but is smooth and could also be polished. Macroscopic recesses 52 are not meant here and may be provided.

Finally, it should be mentioned that the plateau section 4 also has surface regions 60 that are not in contact with tibial bone tissue, which form the circumferential regions of the plateau section 4 and are in contact with soft tissue when implanted. These surface regions 60 are smooth and may be provided with a further coating 62 which further reduces the surface roughness of these smooth surface regions 60, as explained in detail in the introductory description and the referenced prior art.

1. A tibia implant for joint replacement, the tibia implant being made of metal alloy and comprising:
a plateau section; and
an anchor section,
the plateau section comprising a first side configured to face a tibia and a second side configured to face away from a tibia;
the plateau section further comprising a bearing region on the second side for articulating condylar joint surfaces, the bearing region provided on a side facing away from the tibia;
the anchor section being pin-shaped or keel-shaped,
the anchor section projecting from the first side of the plateau section and extending away from the first side in an axial direction,
the anchor section being insertable in the axial direction into a channel prepared in the tibia,
the plateau section having a first surface structure that is a three-dimensional, porous, open-pore surface structure on the first side,
the first surface structure configured to contact tibia bone tissue with bridges, webs, or wall regions that are grippable from in the axial direction,
the first surface structure having a first roughness,
the anchor section comprising a first axial anchor region on a circumferential side with a second surface structure adjacent to the plateau section, the second surface structure being free of undercuts in a radial direction, with a second roughness that is lower than the first roughness, and
the anchor section further comprising a second anchor region that is axially free-ending and has a smooth surface in the axial direction adjacent to the first axial anchor region.

2. The tibia implant according to claim 1, wherein the first surface structure has a depth extension extending from a surface enclosing the first surface structure of at least 1.5 mm.

3. The tibia implant according to claim 1, wherein:
the first surface structure is formed by a contiguous web structure forming a three-dimensional lattice, and
the contiguous web structure has a web diameter of at least 0.5 mm.

4. The tibia implant according to claim 1, wherein the first surface structure has a pore size of at least 0.8 mm.

5. The tibia implant according to claim 1, wherein the second surface structure of the first axial anchor region has a depth extension extending from a surface enclosing the second surface structure of at least 0.2 mm.

6. The tibia implant according to claim 1, wherein the second surface structure of the first axial anchor region has structures rising from a base area of the first axial anchor region.

7. The tibia implant according to claim 1, wherein the second surface structure of the first axial anchor region has rising structures that meander back and forth when viewed in the radial direction.

8. The tibia implant according to claim 7, wherein the rising structures comprise a plurality of flat faceted areas that are adjacent to one another via edges.

9. The tibia implant according to claim 7, wherein the rising structures delimit flat or smooth regions of a base area between the rising structures.

10. The tibia implant according to claim 7, wherein the rising structures comprise a first rising structure and a second rising structure adjacent to the first rising structure, the first rising structure and the second rising structure touching one another and delimiting a less elevated area between the first rising structure and the second rising structure.

11. The tibia implant according to claim 7, wherein the rising structures are spaced apart from one another and delimit strip-shaped contiguous areas between the rising structures.

12. The tibia implant according to claim 1, wherein the second surface structure of the first axial anchor region has a plurality of individual island-like rising structures spaced apart from one another.

13. The tibia implant according to claim 1, wherein the tibia implant is made of titanium or titanium alloy.

14. The tibia implant according to claim 1, wherein the plateau section further comprises smooth surface regions configured to contact soft tissue or a meniscus replacement part.

15. The tibia implant according to claim 14, wherein the smooth surface regions comprise a surface roughness and a coating at least in some regions, the coating configured to reduce the surface roughness of the smooth surface regions.

16. The tibia implant according to claim 15, wherein the coating comprises a ceramic surface.

17. The tibia implant according to claim 15, wherein the coating is multi-layered and bonded to the smooth surface regions via an adhesion-promoting layer.

18. The tibia implant according to claim 15, wherein the coating comprises layers based on at least one of:
cobalt-chromium,
chromium nitride,
chromium carbonitride, or
zirconium nitride.

19. The tibia implant according to claims 15, wherein the coating comprises:
a top layer based on zirconium nitride, and
inner layers based on chromium nitride or chromium carbonitride.

20. The tibia implant according to claim 1, wherein the bearing region is configured for attachment to a meniscus replacement part.
